(19) United States
(12) Patent Application Publication
(10) Pub. No.: US 2015/0165072 A1
(43) Pub. Date: Jun. 18, 2015

(54) CONTRAST IMAGING APPLICATIONS FOR LANTHANIDE NANOPARTICLES

(71) Applicant: University of Iowa Research Foundation, Iowa City, IA (US)

(72) Inventors: Jose G. Assouline, Iowa City, IA (US); Sean K. Sweeney, Iowa City, IA (US)

(21) Appl. No.: 14/415,041

(22) PCT Filed: Aug. 6, 2013
(86) PCT No.: PCT/US13/53834

§ 371 Date: Jan. 15, 2015

Related U.S. Application Data
(60) Provisional application No. 61/679,941, filed on Aug. 6, 2012.

(51) Int. Cl. A61K 49/22; A61B 6/00; A61B 5/05; A61K 49/04; A61B 5/00; A61M 5/00; A61B 5/055; A61K 49/18; A61B 8/08; A61M 31/00

(52) U.S. Cl. CPC A61K 49/222; A61B 8/481; A61B 6/481; A61B 5/0515; A61B 8/0841; A61M 31/005; A61B 5/0044; A61B 5/0042; A61M 5/007; A61B 5/055; A61B 5/0095; A61B 5/0035; A61B 5/0037; A61B 6/4417; A61K 49/1818; A61K 49/04

(57) ABSTRACT

The invention provides methods, devices and compositions that employ EuGd203 nanoparticles, e.g., for imaging. The invention provides for devices having lanthanide doped gadolinium nanoparticles and uses for lanthanide doped, for instance, europium doped, gadolinium (e.g., Eu—Gd203) nanoparticles in sequential imaging. For example, Eu—Gd203 nanoparticles are employed as multimodal/sequential contrast agents for combinations of MRI, CT, ultrasound, and photoacoustics or other imaging modalities such as optical imaging. The invention provides a method comprising administering, for instance, injecting, a composition comprising lanthanide doped gadolinium nanoparticles into a subject, e.g., a human; applying ultrasound, laser pulses, x-rays or a magnetic field, or sequentially applying two or more of ultrasound, laser pulses, x-rays or a magnetic field, or sequentially applying two or more of ultrasound, laser pulses, x-rays or a magnetic field, to the subject; acquiring signals from the nanoparticles; and reconstructing an image from the acquired signals.

### CROSS-REFERENCE TO RELATED APPLICATIONS

This application claims the benefit of the filing date of U.S. Application Ser. No. 61/679,941, filed on Aug. 6, 2012, the disclosure of which is incorporated by reference herein.

### BACKGROUND

Contrast echocardiography is a particular niche in the larger ultrasound imaging modality, in which sound waves are transmitted through tissue and images are formed based on the timing of echoes returning to the transducer. When imaging the heart in echocardiography, contrast agents are sometimes used to highlight certain features, particularly when the patient presents with obstacles to non-contrast echocardiography, such as obesity and lung disease. Three echocardiography contrast agents are currently approved and used clinically. Those agents consist of various polymers encapsulating high molecular weight gases (Chelliah and Senior, 2009) and are typically in the 1-5 μm size range, which makes them small enough to traverse capillaries while being large enough to easily generate an echo. The gases have a dramatically slower speed of sound compared with soft tissue, giving rise to a greater contrast.

For example, Definity® is a perflutren lipid microsphere composed of octafluoropropane encapsulated in an outer lipid shell. The lipid shell is composed of hexadecanoic acid, monosodium salt and inner salt. For proper use, Definity® requires activation by warming it to room temperature and shaking for 45 seconds using a Vialmix®. Once mixed, 1 milliliter of Definity® contains about 1.2×10¹⁰ lipid microspheres and 1.1 mg octafluoropropane. After activation and intravenous injection, Definity® provides contrast enhancement of the endocardial borders during echocardiography.

Generally, nanometer-scale contrast agents are not used in ultrasound because they are much smaller than the smallest attainable spatial resolution of the ultrasound transducer. However, some nanometer-scale echocardiography contrast agents are being used experimentally. Casciaro et al. (2010) tested various diameters (160, 330, and 660 nm) of silica nanobeads. Using agarose gel plates as phantoms, a signal could be observed for silica concentrations up to 0.8% and the particles could also be automatically detected using RF signal analysis (Casciaro et al., 2010). Other solid nanoparticles for ultrasound imaging include iron oxide particles (Bara et al., 2006) and gold nanoparticles (Mallidi et al., 2009).

The success of an imaging modality is based on a variety of factors and so it is difficult to find a contrast agent suitable for more than one imaging modality.

### SUMMARY OF THE INVENTION

The invention provides for devices having lanthanide doped gadolinium nanoparticles and uses for lanthanide doped, for instance, europium doped, gadolinium (e.g., Eu—Gd₂O₃) nanoparticles in sequential imaging. In one embodiment, the nanoparticle has a core of gadolinium oxide, e.g., up to about 30 to about 40 nm, such as about 35 nm, in diameter, which may in one embodiment be prepared by chemical vapor deposition. The gadolinium oxide is doped with a lanthanide, e.g., europium, terbium or ytterbium, at a proportion greater than zero and up to about 20 at. %. The resulting lanthanide doped gadolinium nanoparticles may then be functionalized with one or more organic molecules, e.g., so as enhance their biocompatibility. For example, those materials may include organic polymers such as poly(ethylene glycol) (PEG), polyacrylate or amino acids (glycine, glutamine, glutamate, lysine/poly-L-lysine, etc.). In one embodiment, the nanoparticles may be functionalized with proteins (such as ligands to cell surface receptors, growth factor receptors, and the like) for cell-specific labeling and uptake.

In one embodiment, Eu—Gd₂O₃ nanoparticles are employed as multimodal/sequential contrast agents for combinations of MRI, CT, ultrasound, and photoacoustics or other imaging modalities such as optical imaging. For instance, the fluorescence of Eu alone is about 10,000 fold greater than other fluorophores employed to intracellularly label cells for subsequent in vivo tracking (e.g., fluorescein or rhodamine). In one embodiment, the administration of a single dose of the nanoparticles is employed in conjunction with sequential imaging, for example, MRI and/or CT and photoacoustics, echography and/or ultrasound imaging, of a mammal such as a human. In one embodiment, europium-doped gadolinium nanoparticles are used for sequential imaging, moving from low-resolution, high-throughput modalities such as ultrasound or photoacoustic imaging, to higher resolution modalities such as MRI and CT. Such an approach is time saving, cost saving and has less of an impact on the environment. For example, in contrast to the case where a patient needs multiple scans of differing modalities and multiple contrast agents, and each contrast agent must be sterilized, packaged and administered with its own set of materials, the present invention provides for one agent for all modalities and the materials needed for manufacturing, packaging and administration are reduced by a factor of 2 or 3. This also results in a lower impact on the environment and the hospital reduces biohazardous waste disposal. Moreover, unlike chelates of gadolinium, gadolinium oxide nanoparticles do not release free gadolinium ions into the bloodstream.

For example, the invention provides a method comprising administering, for instance, injecting, a composition comprising lanthanide doped gadolinium nanoparticles into a subject, e.g., a human; applying ultrasound, laser pulses, x-rays or a magnetic field, or sequentially applying two or more of ultrasound, laser pulses, x-rays or a magnetic field, to the subject; acquiring signals from the nanoparticles; and reconstructing an image from the acquired signals. In one embodiment, the method includes acquiring image data of images at a target region in the subject in a predetermined sequence and optionally performing, using a processor, predetermined image processing the image data.

The invention provides lanthanide doped gadolinium nanoparticles having a diameter of about 5 nm to about 200 nm, about 10 nm to about 100 nm, about 20 nm to about 70 nm, or about 30 nm to about 50 nm. In one embodiment, the invention provides europium doped gadolinium nanoparticles about 5 nm to about 200 nm, about 10 nm to about 100 nm, about 20 nm to about 70 nm, or about 30 nm to about 50 nm.

In one embodiment, the lanthanide doped gadolinium nanoparticles have the lanthanide at a proportion of about 1 at. % to about 20 at. %, about 5 at. % to about 15 at. %, or about 7 at. % to about 10 at. %.

Ultrasound is often used as a guide for deployment of devices, e.g., a catheter, in minimally invasive techniques. For instance, a catheter is fed through an artery to a site for a procedure using an ultrasound transducer on the skin to note the location of the catheter tip. The tip itself, or the material being delivered, such as an implantable device, e.g., stent, or tissue, can be labeled with the nanoparticles for improved detection and more precise, e.g., local, delivery. Thus, the particles can be used as a guiding strategy for interventional medicine. Moreover, local delivery of the particles may allow for the use of lower amounts for effective contrast. In one embodiment, the nanoparticles are delivered with ultrasound guided techniques, using a delivery device such as a catheter which is subsequently withdrawn, then traced using a higher-resolution modality such as MRI or CT, without the addition of any other materials. In another embodiment, the nanoparticles are incorporated into a device, e.g., the body of a catheter or a stent, so that device placement can be monitored using ultrasound.

Also provided are kits. In one embodiment, the kit has a delivery device, e.g., a syringe or catheter, and a receptacle having one or more doses of the lanthanide doped gadolinium nanoparticles, for instance, in a physiologically compatible fluid. In one embodiment, the kit has a delivery device, e.g., a catheter or stent, embedded or coated with the lanthanide doped gadolinium nanoparticles.

In addition, the lanthanide doped gadolinium nanoparticles may be used to label tissues ex vivo, e.g., tissues for implantation such as a heart valve, thereby allowing for those tissues to be at least temporarily tagged.

### BRIEF DESCRIPTION OF FIGURES

FIG. 1. Schematic of process to prepare Eu—Gd₂O₃ nanoparticles.

FIG. 2. Characterization of nanomaterials using photoluminescence (PL). Data was obtained at several temperatures from 30 K up to 297 K for RD-136 (A), RD-147 (B), RD-154, and RD-155, using an excitation wavelength of 325 nm (UV). All of the emission wavelengths are near 450 nm (blue), with the peaks appearing sharper at higher temperatures. The second set of particles, RD-154 (10% Eu—Gd₂O₃,C) and RD-155 (4% Li/5% Eu—Gd₂O₃, D), have much taller, sharper PL emission peaks than the earlier specimens, with a primary peak at about 625 nm (red-orange light) and a substantial secondary peak at about 710 nm (deep red light). The amino-acid functionalized particles were characterized by measurement of hydrodynamic diameter using dynamic light scattering (DLS, E) in pure water. This showed diameters of 33 nm for glycine-, TEGO-, and PAA-functionalized particles, and 35 nm for glutamine-functionalized particles.

FIG. 3. Viewing the same drop of 10% Eu—Gd₂O₃ particles (RD-154) on a slide under brightfield (left) and UV exposure (right). In large aggregates, these particles appeared to emit a faint red signal that could be seen under the microscope, but the signal was not seen in smaller concentrations, or in the other sample, RD-155 (lithium-europium co-doped Gd₂O₃).

FIG. 4. A single slice of raw MR images containing four masses of 0.5% Eu—Gd2O3. TE=6 ms (left), TE=14 ms (center) and the result of subtracting the two scans followed by background subtraction and 3D rendering with pseudocoloring (right). Regions near the edges of the gadolinium pellets show the brightest in the subtracted image. The tubes contain 50 μg (A), 250 μg (B), 1 mg (C), and 2.5 mg (dD particles in 500 μL PBS.

FIG. 5. Pseudocolor, difference-subtracted MRI images showing boluses of varying sizes of 5% Eu—Gd₂O₃ particles in 0.5 mL microcentrifuge tubes with PBS as the supernatant. From left to right, the masses of particles are 50 μg, 250 μg and 1 mg, followed by a PBS control. The 1 mg pellet appears to have been disturbed, making subsequent measurements more difficult.

FIG. 6. Pseudocolor 3D rendering of the difference subtracted processed MRI of Eppendorf tubes containing the indicated concentrations of nanoparticles. These particles were maintained in a homogenous suspension in highly viscous type I collagen, making the differences easier to visualize. The average value of the entire volume was plotted, normalized to the collagen control (collagen=1). The plots followed a more linear trend and, as with the previous plots, the europium-doped gadolinium particles gave the highest signal.

FIG. 7. R2 relaxivities for various concentrations of gadolinium suspended in collagen and scanned at several echo times (TE=4, 6, 8, 10, 12, 14, 16, 18, and 20 ms). The plotted best-fit line shows the calculated R2 relaxivity value for the particles to be 3.6 s-1·mM-1.

FIG. 8. 3D rendered transverse section of MRI showing the 3 injection sites near the surface of the brain (red ellipses, A), and the injection sites merging together in the deeper tissue (B).

FIG. 9. Surfaces rendered from manual segmentation of injection sites (dark blue, A, and light blue, B) and the contralateral sides (green, A, and red, B) of mouse brain 1 hour (A) and 48 hours (B) after injection. Comparative diffusion tensor tractography (C and D) using a published mouse brain diffusion tensor dataset. Tracts are shown to indicate fibers involved in a shallow injection (D) as well as a deeper injection (E). Volume of interest (VOI) analysis comparing volume (E) and grayscale intensity (F) over time for left hemisphere (injection sites) and right hemisphere (contralateral side).

FIG. 10. In vivo MRI scans of mouse brain following 3 injections, 1 μL each, of amino acid functionalized nanoparticles. Two scans, one hour (left) and 48 hours (center) following injection were put into registration using MIPAV and put through pixelwise subtraction in ImageJ. The resultant difference image, with maxima in red, is overlaid onto the original one hour image to show regions of greatest change.

FIG. 11. Various concentrations of RD154 (10% Eu—Gd₂O₃) in 1% agarose gels measured by 30 MHz ultrasound, with the regions of interest enclosed in red boxes. PBS (negative control, A), 100 μg/mL (B), 250 μg/mL (C), 500 μg/mL (D), 1 mg/mL (E), and Definity® (F), with a hexafluoropropane concentration of about 1.1 mg/mL when mixed. Plot of average grayscale values of 10% Eu—Gd₂O₃ compared with commercially available Definity® (G).

FIG. 12. Ultrasound images of 250,000 non-labeled mesenchymal stem cells in 1% agar (A) and 250,000 cells labeled with 200 μg/mL 10% Eu—Gd₂O₃, (B) with red boxes to indicate the layer containing cells. Comparison of average grayscale value of each specimen (C).

FIG. 13. 3D reconstruction of an ex vivo heart injected with 200 mg 10% Eu—Gd₂O₃ embedded in 1% agar, and scanned at 30 MHz using a short axis cine loop recording while moving the probe at a constant speed across the specimen. Showing the exterior surface of the whole heart (A) with injection site highlighted (red circle), and pseudocolor 3-D rendering showing clipped plane view of the injection site near the base of the ventricles (B).

### DETAILED DESCRIPTION

### Imaging and Contrast Agents

Nuclear imaging, magnetic resonance imaging (MRI), magnetic resonance spectroscopy, computed tomography (CT), ultrasound (US), bioluminescence, fluorescence imaging (optical imaging), and photoacoustic imaging are employed for imaging in vivo. The success of an imaging modality depends on a combination of various factors. Along with the issues of biocompatibility, toxicity and probe stability, another challenge associated with the use of various imaging modalities is to achieve a high contrast signal over nearby tissues. Each modality has certain advantages and/or disadvantages. For instance, Magnetic Resonance Imaging (MRI) in general has low sensitivity, relatively long imaging times, and uses large amounts of injected contrast agents. However, MRI provides high image resolution and exquisite soft tissue contrast for revealing tissue morphology and anatomical details.

Certain modalities, such as Positron Emission Tomography (PET) and Single Photon Emission Computed Tomography (SPECT), employ radioisotopes, which is less desirable than non-radiolabeled imaging agents. PET can be employed with a wide range of radiolabeled tracers with varied half life, and provides for high resolution, high contrast and high sensitivity and specificity. Although Single Photon Emission Computed Tomography (SPECT) is low cost in comparison to PET tracers and can employ multiple probes, drawbacks of SPECT include photon attenuation, scatter, and geometric blurring caused by radionuclide collimators dimensions.

Optical imaging is minimally invasive and does not employ ionizing radiation, is sensitive in the pico-molar range, is low cost, and is highly sensitive and specific for targeted molecules.

Photoacoustic imaging (PAI), also called optoacoustic or thermoacoustic imaging, combines spectral selectivity by laser light with the high resolution of ultrasound imaging. PAI techniques include photoacoustic tomography (PAT) and photoacoustic microscopy (PAM). PAT utilizes either an ultrasound detector array or a single scan detector and an inverse algorithm in reconstructing cross-sectional or three-dimensional images PAM typically uses raaster-scanned focused ultrasonic detector coupled with confocal optical illumination. PAI takes advantage of the high optical contrast of biological tissue. Thus, PAI is suitable for monitoring both endogenous and exogenous optical contrast agents.

Nanoparticle-based contrast agents vary in diameter from 1 nm to several hundred nanometers. Nanoparticles designed for high photoacoustic contrast can be classified into one of two major types based on the physical mechanism of light absorption: particles based on surface plasmon resonance (SPR) and dye-containing nanoparticles. For SPR applications, nanoscale metallic films most commonly gold, are deposited onto the nanoparticle surface. Dye-containing nanoparticles utilize a high payload of near infrared (NIR) organic dyes to enhance optical absorption, e.g., indocyanine green (ICG) has been encapsulated within nanoparticles and used as a contrast enhancer for PAI. The use of nanoparticle-based contrast agents greatly extends PAI applications because it allows PAI to image deeper within tissue with enhanced contrast. Nanoparticles are usually designed with peak absorption in the NIR region, where optical attenuation of tissue is relatively low, affording deep light penetration.

Four kinds of nanoparticles are used in PAI. Nanoshells usually consist of a dielectric core coated by a conductive, nanometer-thick metallic shell, usually gold. In particular, nanoshells have been engineered with absorption peaks ranging from 600 to 900 nm. A second kind of nanoparticle is a gold nanorod whose absorption mechanism is based on SPR. Another kind of nanoparticle is a gold nanocage. Those are prepared by a simple galvanic replacement reaction between Ag nanocubes and HAuCl₄. By adjusting the amount of HAuCl₄ added, the SPR peaks of the resultant Au nanocages can be tuned throughout the visible and NIR regions.

Other SPR nanoparticles have even more complex shapes, such as multipods, star shapes, lumps, etc. Unlike most traditional optical imaging techniques, in PAI, the signal from photoacoustic effect is proportional to optical absorption.

Current ultrasound agents can be divided into two main classes: 1) microbubble based contrast agents and 2) non microbubble based contrast agents. Microbubbles are gas-liquid emulsions consisting of a gaseous core surrounded by a shell and are usually 1 to 4 microns in size. Different types of contrast microbubbles have been synthesized by combining different shell compositions such as albumin, galactose, lipids or polymers, with different gaseous cores such as air, or high molecular weight gases (perfluorocarbon, sulfur hexafluoride or nitrogen). Imaging using Ultrasound/Microbubbles (MB) is relatively inexpensive, transportable, and is a real-time imaging modality.

Non microbubbles based contrast agents consist of either submicron or nano sized particles. These particles consist of either liquid or solid colloids that range in size between 10 and 1000 nanometers. Non microbubble based contrast agents are advantageous over microbubbles in terms of their ability to enter the extravascular space providing the opportunity to image targets beyond the vascular compartment. However, most of the non microbubble based contrast agents cannot be detected individually by ultrasound imaging due to their poor inherent acoustic reflectivity.

Based on their composition and size, different types of sub micron or nano sized particles have been synthesized for ultrasound imaging: Echogenic liposome, perfluorocarbon emulsion (PFC) nanodroplets, nanobubbles, microbubbles, and solid nanoparticles. Echogenic liposomes consist of a lipid bilayer with an aqueous core, and air pockets within the lipid bilayer can generate acoustic reflexivity. Liposomes can range from 20 nm to 10 μm. PFC nanodroplets are liquid-liquid emulsions consisting of a liquid perfluorocarbon coencapsulated by a phospholipid monolayer. PFC nanodroplets are about 200 to 400 nm in diameter and can be vaporized into echogenic gas-bubbles following administration of acoustic energy. Nanobubbles are gas-liquid emulsions enclosed by a biodegradable polymer such as polylactic acid (PLA). Nanobubbles, such as a PLA nanobubble of about 40-200 nm, can fuse into echogenic microbubbles at a target site. In general, microbubbles are 1-4 μm and are surface modified, such as one modified with a polyethylene glycol (PEG) polymer, to prevent aggregation. Microbubbles are highly echogenic and the most commonly used contrast agent for molecular ultrasound imaging. Solid nanoparticles of about 20-100 nm, such as amorphous solid particles containing silica or iron oxide particles, contain gas pockets in their pores or fissures, increasing their echogenicity.

### Multimodal Agents of the Invention

Multimodal particles are particularly useful to enhance signals in multiple scanning modalities. For example, a bolus of particles or cells labeled with particles is introduced to a host organism, e.g., a mammal, in a single dose and the recipient/subject is sequentially subjected to different scanning modalities (e.g., Echo, MRI and/or CT). Some of the faster/less time consuming scans (e.g., photoacoustic) may be used as a guide for more comprehensive scans (e.g., MRI and/or CT). Resulting image analysis for the various analyses can then be compared for more discriminated evaluation. The particles of the invention are quite useful in such sequential procedures as they are very stable over time unlike traditional contrast agents which are for only one modality and are active for only short periods of time after reconstitutions (for instance, for minutes or a couple of hours.

Gadolinium (Gd) has gained popularity as an MRI contrast agent because, like iron oxide, it affects large changes in the local magnetic fields where it is present. By virtue of the fact that it has 7 unpaired electrons in its outer shell, it interacts very efficiently with surrounding protons. If the same specimen is scanned at two different echo times, the changes in field effects between the two scans is larger relative to the differences between background materials. Therefore, a simple subtraction of one image at one echo time from the other further enhances the tracing of the material.

The present invention provides lanthanide dopes Gd containing nanoparticles that are useful as multimodal agents in imaging such as MRI, CT, ultrasound and photoacoustic imaging.

### Magnetic Resonance Imaging (MRI)

Magnetic resonance imaging uses small variations in the magnetic field arising from differing proton spin densities ρ(x,y) in tissue to generate its images. Briefly, the grayscale value at each pixel in a slice of an MR image is the 2-dimensional inverse Fourier transform of that slice's k-space, or frequency domain s(t). The radio frequency (RF) data encoded in the frequency domain is collected when small perturbations are made in the larger magnetic field of the MR scanner using smaller gradient coils that vary over time as Gₓ(t) and G_{y}(t). The general equations describing the signal are

$$s ( t ) + {\int }_{- ∞}^{∞} {\int }_{- ∞}^{∞} ρ ( x , y ) {e}^{- j 2 π [ {k}_{x} ( t ) x + {k}_{y} ( t ) y ]} d x d y , \\ where$$

$${k}_{x} ( t ) = {\int }_{0}^{t} \frac{γ}{2 π} {G}_{x} ( τ ) d τ , \\ and$$

$${k}_{y} ( t ) = {\int }_{0}^{t} \frac{γ}{2 π} {G}_{y} ( τ ) d τ .$$

The MR signal of a specific tissue can also be described by its parameters (T₁, T₂, T₂* relaxation times) and the parameters of the scan (repetition time TR, echo time TE, and/or flip angle α). After a material is magnetized with a certain flip angle α, the magnetic field in the longitudinal axis M_{z} decays (relaxes) with time constant T₁, and varying the time between pulses (repetition time T_{R}), tissue with different T₁ relaxation times show up with different levels of intensity in the reconstructed image. This is known as a T,-weighted image. In a T₂-weighted image, the echo time T_{E} (the time between the pulse and the midpoint of signal readout) is used to generate differing intensities between tissues of different T₂/T₂* relaxation times (the time constant of relaxation or “de-phasing” in the transverse plane, or M_{xy}).

Besides varying T_{R}/T_{E} on the scanner to achieve the desired contrast, additional contrast can be achieved by using one of several contrast agents. MRI contrast agents are ferromagnetic, paramagnetic, or superparamagnetic materials which interact with the protons present in the surrounding medium, thereby altering the apparent T₁ or T₂ relaxation time. The effect of contrast agents on the observed T₁ or T₂ value is given by the equation

$$\frac{1}{{T}_{obs}} = \frac{1}{{T}_{tissue }} + r [ contrast ] ,$$

where T_{obs} is the observed T₁ or T₂ value, Tₜᵢₛₛᵤₑ is the actual T₁ or T₂ relaxation time of the tissue being scanned, r is the r₁ or r₂ relaxivity of the contrast agent, measured in s⁻¹·mM⁻¹, and [contrast] is the molar concentration of the contrast agent (Lauffer, 1987).

Most ferromagnetic contrast agents involve the use of superparamagnetic iron oxide (SPIO), which perturbs the tissue's local magnetic field, causing a change in T₂ or T₂* relaxation time.

In MRI, the signal-to-noise ratio (SNR) is proportional to the magnetic field, the voxel size, and the square root of total scan time. Compared to the above scan parameters, the magnetic field of a clinical scanner is reduced by a factor of 3, the voxel size is increased by a factor of about 2, and the scan time is reduced by as much as 8 times (from up to 4 hours to about a half hour). Therefore, the reduction in signal can be estimated as 2/(3\sqrt{8}), or about 4.2 times smaller. This can be approximately balanced out by increasing the number of labeled cells from the 5×10⁴ that was detected in the above scans to about 2×10⁵. For both of these estimations, the actual thresholds are likely to vary from these estimates, and for different tissues.

### X-Ray Computed Tomography (CT)

Unlike MRI in which the contrast is derived from magnetic properties of tissue, CT images are essentially based on the density of the tissue in the path of the x-rays. In summary, x-ray photons at a known energy are projected towards the patient and detected on the other side. The simplified equation of intensity of the photons striking the detector is given by the relationship

I(x)=I₀e^{-μX},

where I₀ is the initial intensity and μ represents the attenuation coefficient of the material (a function primarily of tissue density). In order to generate a multislice CT image, this principle is expanded to a 2 dimensional detector which can be rotated around the body. Many 2-D projections are compiled into a 3-D image according to the equation

I^{θk}=I₀e^{-Σ}^{ᵢⱼ}^{w}^{ᵢⱼ}^{^{θk}}^{μ}^{ᵢⱼ},

where I^{θk} is the intensity data for detector position k and angle θ, wᵢⱼ is a weighting value for position (i,j) on the detector at position k and angle θ, and μᵢⱼ is the attenuation of the material at position (i,j).

In x-ray CT, contrast agents are effective if they have an ability to greatly change the x-ray opacity of the tissue of interest. Therefore most of the early contrast agents were based on heavy elements such as iodine and barium. Because of toxicity concerns, these agents have evolved over time, and other contrast agents based on electron-dense metals have also been studied, and are well reviewed by Yu/Watson. Of the heavy metal contrast agents, those based on gold, bismuth and gadolinium appear to be the most studied.

In CT imaging, the signal-to-noise ratio is a function of the number of x-ray photons that reach the detector for each pixel. This is affected by the x-ray source voltage, the properties of the collimator which reduces noise from scattered photons, and the spatial resolution. The source voltage remains the same regardless of whether the system is a micro-CT or larger clinical CT scanner. The properties of the collimator are unknown, but are assumed to be similar for both scanner types. The biggest difference between the scanner types is the spatial resolution, which can be under 50 μm for micro-CT, and around 0.5 mm for clinical scanners. Therefore, in a worst-case scenario, for a volume of labeled cells to be detected within a voxel that is 10 times larger, the volume should be 10 times larger as well. It is reasonable then to assume that a mass of 17 million cells would be detected in the lungs of a human subject.

### Ultrasound

Ultrasound is perhaps the fastest and safest way to obtain in situ images, as it requires only a few seconds of preparation with ultrasound gel and produces no ionizing radiation. The drawback is that the spatial resolution does not approach what is possible in CT or MRI at this time. In this modality, a piezoelectric transducer produces sound at high frequencies (typically between 2 and 15 MHz for clinical applications and up to 40 MHz or more for research applications) and generates an image based on the timing of echoes returning to the transducer. Echoes are generated when the propagated sound wave strikes an interface between volumes with differing acoustic impedances (Z) and part of the sound wave reflects back to the transducer. Acoustic impedance is defined as

Z=ρc,

where ρ is the density and c is the speed of sound in the tissue⁷⁵. At the interface between two tissues, the reflectance coefficient (R) describes the fraction of sound energy that will be reflected back to the transducer. The remaining fraction continues propagating deeper into the tissue where it may strike another interface. R is related to the acoustic impedances of the two tissues at the interface (Z₁ and Z₂) according to the equation

$$R = {[ \frac{{Z}_{1} - {Z}_{2}}{{Z}_{1} + {Z}_{2}} ]}^{2} .$$

These principles are applied to the generation of ultrasound images. In A-mode imaging, one transducer is used to plot all the tissue boundaries along one axis as a function of time. One application of A-mode imaging is tracking opening and closing of heart valves or movement of a ventricle during the heart cycle in echocardiography. In B-mode imaging, an array of transducers is coordinated to form a 2D image. This may be the most common way ultrasound is used, and includes fetal sonography among other applications. Newer ultrasound systems are capable of Doppler mode, in which frequency shifts in the sound wave are used to calculate blood flow through arteries, and even 3D ultrasound, in which the transducers are swept across many 2-D fields in rapid succession to generate a 3-dimensional image.

### Exemplary Imaging/Image Processing Parameters

### MRI

Eight 500 μL Eppendorf tubes were filled with 300 μL of varying concentrations of nanoparticles (1% Eu—Gd₂O₃:150 and 500 μg/mL; 0.5% Eu—Gd₂O₃:50, 150 and 500 μg/mL) and suspended in a highly viscous suspension of type I collagen derived from rat tail tendon (this would prevent formation of a pellet during the MRI scan). In addition, a tube containing collagen only was added as a negative control. Tubes were placed in the 4.7 Tesla Varian® MR scanner and scanned to determine the r₂ relaxivity through a sequence of T₂-weighted scans (relaxation time T_{R}=35 ms, echo time T_{E}=4, 6, 8, 10, 12, 14, 16, 18, and 20 ms). In addition, pairs of T₂* gradient echo scans (T_{R}=35 ms and T_{E}=6 and 14 ms, 256 slices and a voxel size of 148 μm per side) were performed.

To calculate the r₂ relaxivity of the gadolinium oxide particles, the program “MRI Analysis Calculator” was downloaded from the ImageJ website (http://rsbweb.nih.gov/ij/plugins/mri-analysis.html). This program requires as input an image stack containing the same slice of data at each of the different echo times. It then calculates the T₂ value at each pixel in the slice using a Simplex best-fit algorithm to solve the following equation for T₂:

Sₙ=S₀e^{(-T}^{_{En}}^{/T}^{₂₎},

where Sₙ is the signal value at the pixel at each echo time T_{En} and S₀ is the initial magnetic field (a constant).

After obtaining the T₂*-weighted scans, post-processing was done in both ImageJ and MIPAV. In ImageJ, the images from the 2 different scans were subtracted, and the difference saved as a third image. This difference image was also put through the automated background subtraction algorithm in ImageJ, with a radius of 50 pixels, to smooth the background noise. In MIPAV, volumes of interest (VOIs) of each of the tubes in both the T_{E}=6 ms and the difference image were selected by manual segmentation. Additional VOIs were selected for PBS controls. VOI volumes and average MR intensities were obtained and saved to a separate file for further analysis.

The standard deviations of the average intensities, when measuring the entire volume of the tube, were similar to those of other scans, so it was deemed that the dispersion was maintained reasonably well for the duration of the scan. When the difference-subtracted images were normalized to collagen and measurements made, the resulting plots reflected the scans. The signal intensity in europium-doped gadolinium was about 30% higher than collagen at 50 μg/mL, and increased to about 50% higher at 500 μg/mL.

A new T₂-weighted scan at several echo times (4, 6, 8, 10, 12, 14, 16, 18, and 20 ms) was performed so that the r₂ relaxivity value could be calculated and compared with other available particles. Using MATLAB, the original MR images for the tubes were split into individual slices which were reorganized so that each slice contained all the echo times stacked together. One by one, these were processed using the ImageJ plugin “MRI Analysis Calculator”. The calculated T₂ times were reassembled into a single stack so that MIPAV volume-of-interest (VOI) tools could be used to isolate the different concentrations within the scan. The mean T₂ value was calculated for each scan, and a plot of 1/T₂ vs. concentration was made. The plot shows the equation of the best fit line, used to determine the r₂ relaxivity value for the particles, which was 3.6 s⁻¹·mM⁻¹, and with a strong R-squared value of 0.98.

For ex vivo MR imaging, the chest cavity is opened and the inferior vena cava (IVC) is severed. A gravity-fed apparatus containing normal saline with a 22 gauge needle is inserted into the right ventricle of the mouse to clear the blood from the vasculature. Both fluids are set on a shelf approximately 1.5 meters above the benchtop in order to deliver the fluids at a hydrostatic pressure of about 110 mmHg, or roughly the systolic pressure of a normal mouse. After the blood draining from the IVC runs clear, the apparatus is switched to deliver 4% paraformaldehyde. Perfusion fixation is continued until the mouse's tail curled and then went straight, a sign of muscle fibers cross linking (about 10 minutes of flow). Injections are made into the tissues, as are PBS sham injections and needle sticks only as controls. T2*-weighted pulse echo sequences are used for MR imaging.

The organs were stored in 15 mL centrifuge tubes with 4% paraformaldehyde and scanned in the 4.7 Tesla Varian® small animal scanner. After opening the images in MIPAV, each injection site could be observed in the 3D reconstructions of each organ. The volumes of interest were selected and measurements were made: total volume in voxels and in mm³, and average and standard deviation of the intensity value (in arbitrary units). Control volumes of interest were selected as well from normal tissue away from the injection sites. Statistical comparison of two means was performed on the data, and significance (p<0.05) was observed. In the case of the brain, comparison of the injection site to the nearby ventricles did not show a significant difference.

In the case of the heart, 3 injections of 50,000 cells each were made in the same heart, and their intensity value averaged. This average was compared to both normal heart tissue as well as to air bubbles which were trapped in the centrifuge tube, because to the naked eye, these had a similar hypointense value as the injections of cells. In both comparisons, significance was observed.

### CT

For micro-CT imaging, a similar method is used; however, prior to opening the chest cavity, the trachea is exposed, partially cut, and cannulated with a flexible 22 gauge Luer-lok cannula. Through the cannula, MSN particles are delivered to one of the lungs. The lungs are then inflated by connecting the cannula to a source of air pressure for the remainder of the perfusion fixation. The heart/lungs are dissected out as one unit, still under air pressure through the trachea, and dried in a drying oven for several days. Scans are performed at varying voltages and currents.

For both CT and MRI, the freeware medical image processing program MIPAV is used for image analysis. The isolevel selection tool is used to manually segment volumes of interest (VOIs): in MR heart imaging, the injection sites as well as control volumes for myocardium and paraformaldehyde, and in CT lung imaging, the terminal bronchioles containing labeled cells as well as an unlabeled region in the contralateral lung. For each VOI, MIPAV calculates the mean and standard deviation of intensity value and number of voxels, and these figures are used for pairwise statistical analysis using the t-test for comparison of two means with independent samples and unequal variances:

$$t = \frac{( {\overline{x}}_{1} - {\overline{x}}_{2} ) - ( {μ}_{1} - {μ}_{2} )}{\sqrt{( {s}_{1}^{2} / {n}_{1} ) + ( {s}_{2}^{2} / {n}_{2} )}} ;$$

$$v = \frac{{[ \frac{{s}_{1}^{2}}{{n}_{1}} + \frac{{s}_{2}^{2}}{{n}_{2}} ]}^{2}}{[ \frac{{( {s}_{1}^{2} / {n}_{1} )}^{2}}{{n}_{1} - 1} + \frac{{( {s}_{2}^{2} / {n}_{2} )}^{2}}{{n}_{2} - 1} ]} .$$

where n₁ and n₂ are the number of voxels in each VOI, s₁² and s₂² are their respective standard deviations, and are their means and v is the degrees of freedom used in reference to the statistical lookup table.

### Exemplary Cells for Administration

Cells within the scope of the invention, e.g., those which are labeled with the particles described herein, include but are not limited to bone marrow-derived cells, e.g., mesenchymal cells and stromal cells, smooth muscle cells, fibroblasts, SP cells, pluripotent cells or totipotent cells, e.g., teratoma cells, hematopoietic stem cells, for instance, cells from cord blood and isolated CD34⁺ cells, multipotent adult progenitor cells, adult stem cells, embyronic stem cells, skeletal muscle derived cells, for instance, skeletal muscle cells and skeletal myoblasts, cardiac derived cells, myocytes, e.g., ventricular myocytes, atrial myocytes, SA nodal myocytes, AV nodal myocytes, and Purkinje cells. Thus, the cells include embryonic, fetal, pediatric, or adult cells or tissues, including but not limited to, stem cells and precursors (progenitor) cells. For example, the cells can be myocardial cells, bone marrow cells, hematopoietic cells, lymphocytes, leukocytes, granulocytes, hepatocytes, monocytes, macrophages, fibroblasts, neural cells, mesenchymal stem cells, beta-islet cells, and combinations thereof, or cells capable of differentiating into those cells. In one embodiment, the cells are autologous cells, however, non-autologous cells, e.g., xenogeneic or allogeneic cells, may also be employed.

Routes of Administration of Particles or Cells Labeled with Particles

The compositions of the present invention may be administered by any means known in the art. For example, the compositions are suitable for parenteral administration, for instance by intramuscular, subcutaneous or intravenous routes. The compositions of the invention may also be administered subcutaneously, into vascular spaces, or into joints, e.g., intraarticular injection. The local delivery of the compositions can also be by a variety of techniques. Examples of delivery vehicles include catheters, such as an infusion or indwelling catheter, a needle or other device for injection, implantable devices, or site specific carriers.

The compositions suitable for injection or infusion may include sterile aqueous solutions or dispersions or sterile powders comprising the MSNs which are adapted for the extemporaneous preparation of sterile injectable or infusible solutions or dispersions. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

For example, for engraftment, cells are labeled with the particles and then implanted into a recipient (e.g., human or other primate, or other mammal). Alternatively, the nanoparticles are injected as a bolus directly into an organ of interest. Parenteral injections are also envisioned and are warranted for certain applications. Catheter based delivery of the nanoparticles or cells may be employed, e.g., for delivery within the brain with minimal trauma to surrounding structures and so as to avoid to critical cerebral structures, yet allowing for delivery to deep zones. Translumenal catheter based approaches are also envisioned, e.g., for treatment of stroke, chronic neurological diseases or cerebrovascular diseases. Catheters may also be used to deliver nanoparticles or cells, for instance, progenitor cells, having nanoparticles intramyocardially or intravascularly, e.g., via an intracoronary approach. Monitoring of the nanoparticles or cells may be accomplished by photoacoustic (ultrasound), MRI and/or CT imaging.

In addition, microrobots and nanorobots may be employed, e.g., for repairs that are currently being performed laparoscopically. In this approach, nanoparticles are introduced (e.g., via a microrobot or nanorobot), or injected into areas of interests and are activated by creating intermittent acoustic/electric or magnetic fields.

The invention will be further described by the following non-limiting examples.

### Example 1

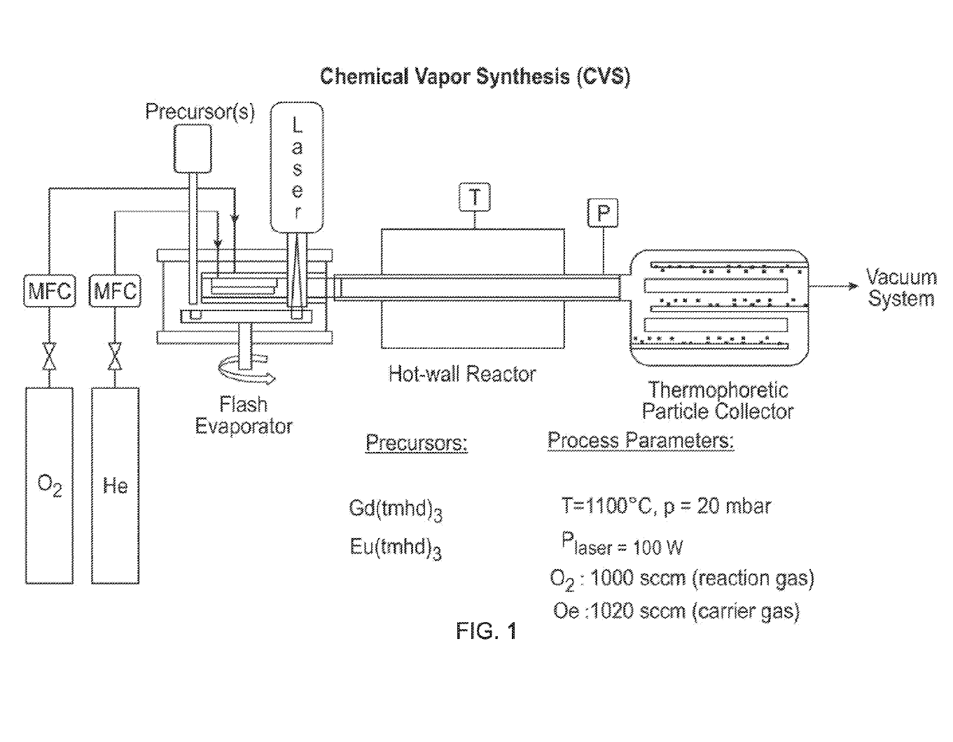

Eu—Gd₂O₃ nanoparticles are synthesized by Chemical Vapor Synthesis (CVS). This process uses solid precursors of Gd(tmhd)₃ and Eu(tmhd)₃. Helium gas at 1020 sccm is used as a carrier and Oxygen at 1000 sccm is used as the reaction gas. The solid precursors are evaporated into the gas phase through the use of a flash evaporator. In this procedure, the solid precursors are dropped into a groove on a rotating wheel that has a 100 W laser on the opposite half of the wheel shining onto the groove. Once the precursors have rotated half way around the rotating disk, the laser causes them to immediately enter the gas phase and mix with the carrier gas and reaction gas. After the flash evaporator, the gas enters a hot-wall reactor operating at 1100° C. and a pressure of 20 mbar. The hot-wall reactor is followed by a thermophoretic particle collector that uses temperature gradients to collect the particles on its walls. The particles are then collected as a powder. See FIG. 1.

Once the nanoparticles are obtained as a powder, they are often converted into a colloidal dispersion for characterization. This is performed by putting the powder into a liquid (e.g., water, an acid or a buffer such as PBS) and then using an ultrasonic horn for mixing. Then using a Zetasizer laser instrument that utilizes dynamic light scattering (DLS) technology the diameter of the particles is calculated. The colloidal dispersion can also be used for determining the photoluminescence emission and excitation spectra using a spectrometer.

### Example 2

### Materials and Methods

### 1.1 Nanomaterial Synthesis and Characterization

Powdered nanomaterials containing various combinations of components (Table 1) were synthesized by the lab of Markus Winterer of the University of Duisburg-Essen (Sandmann et al., 2012), and the photoluminescence was characterized using laser photospectrometry. rhe e particles were first suspended in 70% ethanol to ensure sterility. The ethanol was allowed to evaporate and the particles were suspended in sterile phosphate buffered saline (PBS, Gibco) at a stock concentration of 10 mg mL⁻¹. Immediately prior to their use, the stock suspensions were sonicated using an ultrasonic probe for 5-10 seconds. To observe luminescence, a drop of each type of nanoparticle was placed on a glass slide and covered with a coverslip, and viewed under a 350 nm (ultraviolet) excitation wavelength on the fluorescent microscope.

The 0.5 at. % Eu-doped Gd₂O₃ and 5 at. % Eu-doped Gd₂O₃ were unable to be visualized as luminescent under the microscope. RD-154 has an increased Eu content relative to RD-136 and RD-147. RD-155, has a lithium co-dopant. Amino-acid functionalized particles, AS22(a-d) (Table 1), were prepared and measured for nanoparticle mass, capping agent mass/volume, volume of dispersion, and hydrodynamic diameter using dynamic light scattering (DLS).

TABLE 1

RD-136 (0.5 at. % Eu—Gd₂O₃ as-synthesized)
RD-147 (5 at. % Eu—Gd₂O₃ as synthesized)
RD-154 (10% Eu—Gd₂O₃)
RD-155 (4% Li,5% Eu—Gd₂O₃)

| | | | mass | | |
|---|---|---|---|---|---|
| Sample | Mass | Capping | (capping | Volume | DLS |
| name | (Eu—Gd₂O₃) | agent | agent) | (H₂O) | (diameter) |
| AS-22a | 20.3 mg | L-Glycine | 20.5 mg | 5 mL | 33 nm |
| AS-22b | 20.1 mg | L-Glutamine | 20.1 mg | 5 mL | 38 nm |
| AS-22c | 20.1 mg | TEGO | 800 μL | 4.2 mL | 33 nm |
| AS-22d | 19.9 mg | PAA | 200 μL | 4.8 mL | 33 nm |

### 1.2 Magnetic Resonance Imaging

Eight 500 μL Eppendorf tubes were filled with 300 μL of varying concentrations of nanoparticles (1% Eu—Gd₂O₃: 150 and 500 μg/mL; 0.5% Eu—Gd₂O₃: 50, 150 and 500 μg/mL) and pelleted. In addition, a tube containing PBS was added as a negative control. The tubes were placed in the 4.7 Tesla Varian® MR scanner and scanned using a T2* gradient echo scan with two different echo times (T_{E}=6 ms and T_{E}=14 ms), 256 slices and a voxel size of 148 μm per side.

After obtaining the raw images, post-processing was done in both ImageJ and MIPAV. In ImageJ, the images from the 2 different scans were subtracted, and the difference saved as a third image. This image was also put through the automated background subtraction algorithm in ImageJ, with a radius of 50 pixels, to smooth the background noise. In MIPAV, volumes of interest (VOIs) of each of the pellets in both the T_{E}=6 ms and the difference image were selected by manual segmentation. Additional VOIs were selected for PBS controls. VOI volumes and average MR intensities were obtained and saved to a separate file for further analysis.

To compare relative intensities of raw (not subtracted) MR values to one another, each was divided by the average value of the PBS VOI. This way the PBS voxels would have a value of 1.0, with higher contrast being attributed to voxels that deviate the furthest from 1.0.

Following this experiment, a second set of scans was done in which the particles were suspended in a highly viscous collagen mixture in order to prevent them from settling during the experiment. In this way, the imaging characteristics of more disperse particles could be seen.

### 1.3 Computed Tomography

All 8 Eppendorf tubes containing collagen suspensions previously scanned in MR and the labeled, dried heart (along with an Fe₂O₃-FITC-MSN-labeled mouse lung) were sent to SkyScan for microCT scanning on the SkyScan model 1172 scanner. The specimens were scanned with a source voltage of 59 kV and a current of 167 μA. The final .tiff file contained 1500 slices at a resolution of 1336×2000 and voxels of 7 μm on a side. For viewing using MIPAV, the images were subsampled by a factor of 3 or 6.25, making the voxel sizes 21 or 43.75 μm, respectively. The Eppendorf tube images with 21 μm pixel size were all cropped close to the outer edge of the tube and in the same dimensions across all tubes to facilitate comparison between tubes.

The first analysis performed on the Eppendorf tube standards was to simply subtract the grayscale value of the collagen control (which was found to be 61.2) from the other 7 experimental tubes using ImageJ. Histograms were taken of each image and the means were recorded. The mean value of each image therefore has arbitrary units and represents a combination of intensity and volume arising from only the nanoparticles.

In an attempt to further elicit useful information from these scans, a more precise isolation of the actual nanoparticles was needed. The contrast between the signal and background was sufficiently high in 4 of the images to perform fuzzy c-means segmentation and automatically segment the volume of interest (VOI). In the remaining three images, automatic segmentation was not able to isolate the desired volumes, so the VOIs were manually segmented, and in all 7 tubes, the mean grayscale value and total volume (in pixels) of the VOIs was recorded.

Lastly, the mean grayscale values of the above VOIs were used to estimate the value of the contrast agent in Hounsfield Units, which are defined as:

$$\begin{matrix}\begin{matrix} \\ \begin{matrix}HU = \frac{μ - {μ}_{{H}_{2} O}}{{μ}_{{H}_{2} O}} * 1000 \\ \end{matrix}\end{matrix} \\ \end{matrix}$$

where μ is the mass attenuation coefficient of the material and μ_{H2O} is the mass attenuation coefficient of water. On this scale, then, air has a value of −1000 HU, water is 0 HU, and biological tissues range from around −100 for fat to +400 for bone. Contrast agents vary greatly, but Pietsch et al. (2009) report a value of 385 HU for 10 mg/mL of gadolinium. The CT data for the Eppendorf tubes was fit onto the Hounsfield scale using the assumptions that a) the volume outside the tubes was air (i.e., −1000 HU), and had an average grayscale value of zero, and b) the collagen mixture, with a density approximately equal to water, would be 0 HU and have a mean grayscale value of 61.2. Thus, the equation of the scale was HU=(1000/61.2)*(grayscale value)−1000=16.34*(Grayscale value)−1000.

### 1.4 Echocardiography

Twelve hours prior to preparing the agars, one of two T75 flasks confluent with F015 E6/E7 immortalized mesenchymal stem cells was labeled with 200 μg/mL nanoparticles (RD154, 10% Eu—Gd₂O₃). The following day, a 1% agarose in PBS solution was made, and maintained at 50° C. while the other materials were prepared. The nanoparticles were sonicated for 5-10 seconds, and 20 μL at a concentration of 10 mg/mL was injected into the wall of the left ventricle of a 16 week fetal heart. No visible regurgitation of the injection out of the needle hole could be observed. Next, 5 mL molten agar was poured into each of 7 35 mm dishes as well as about 10 mL into a 25 mL beaker into which the heart would be placed. The nanoparticles were added to the agars to make concentrations of 0, 100, 250, 500, and 1000 μg/mL, and the T75 flasks of labeled and unlabeled cells were trypsinized, fixed in 4% paraformaldehyde and added to the remaining two plates. Approximately 2.5*10⁶ cells were added to each plate.

At the ultrasound scanner, the 30 MHz transducer was used. Each prepared agar was carefully removed from its Petri dish, coated with a layer of ultrasound gel, and scanned. For comparison studies, the gain was held constant at 28 dB; all other scanning parameters were kept constant as well. For the heart, several cine loops in both the short axis and long axis were made which ran the length of the entire organ, and the loops were rendered in 3 dimensions to form a tomographic view of the heart and injection site.

### 1.5 In Vivo MRI

Day 1, morning: an adult Balb/c mouse was anesthetized via intraperitoneal injection of 60 μL ketamine at 50 mg/mL. Once fully anesthetized, it was placed in a stereotaxic frame, the cranium exposed, and a high speed drill was used to open a hole in the cranium, exposing a portion of the left hemisphere of the brain. Through this hole, 3 injections, 1 μL each, were made at the coordinates shown (Table 2). Approximate locations of injections in the brain are indicated (left), and the details of each injection (mass and coordinates) are also indicated (right).

TABLE 2

| | Mass | Medial- | Rostral- | |
|---|---|---|---|---|
| Material | (μg) | Lateral | Caudal | Depth |
| Glycine + 10% | 8.2 | 3.0 mm left | Bregma minus | 1.8 mm |
| Eu—Gd₂O₃ | | of midline | 2.5 mm | |
| Glutamine + 10% | 8.0 | 2.0 mm left | Bregma minus | 3.0 mm |
| Eu—Gd₂O₃ | | of midline | 3.0 mm | |
| TEGO + 10% | 8.0 | 1.9 mm left | Bregma minus | 3.4 mm |
| Eu—Gd₂O₃ | | of midline | 3.6 mm | |

One hour after injection, the mouse was placed in a chamber with isoflurane until unconscious, then positioned in the coil and connected to a steady flow of 3% isoflurane via a nose cone. Two T2*-weighted gradient echo MRI scans (T_{E}=6 and 14 ms, 15 minute scan time each) were collected, along with a short, lower resolution scan for anatomical reference. The scans were repeated with the same parameters 24 and 48 hours after injections.

After the 48 hour scan, the mouse was again anesthetized, its chest cavity opened, and the inferior vena cava cut. The vasculature was perfused with saline until all blood was cleared, then perfused with 15 mL 4% paraformaldehyde to fix all tissues. The brain was excised and stored overnight in 4% paraformaldehyde, then sectioned into 50 mm sections using the Vibratome. The sections were laid out onto a series of slides and allowed to dry.

Prior to staining, the slides were examined under fluorescence microscopy to detect europium. However, the only filter set available with UV excitation was 430 nm (blue) emission. Therefore, the slides were simply viewed under a UV blacklight, which appeared to reveal a faint red streak near the needle tracks.

The MRI image processing algorithm is as follows: raw MRI images were processed using MIPAV and ImageJ. Each day's T_{E}=6 ms scan was used to bring the images into registration. The landmark-least squares algorithm built into MIPAV was used, with the 1 hour scan used as reference, and the 24 and 48 hour scans being transformed. Four landmark points were chosen which could be identified on all images and the transformation matrix was automatically calculated. These matrices were then applied to the corresponding T_{E}=14 ms images, as well as the difference-subtracted images, obtained using pixel-wise subtraction in ImageJ. Once all the images were brought into registration, they were compared in various ways for qualitative and quantitative analysis.

### Results

### Particle Characterization

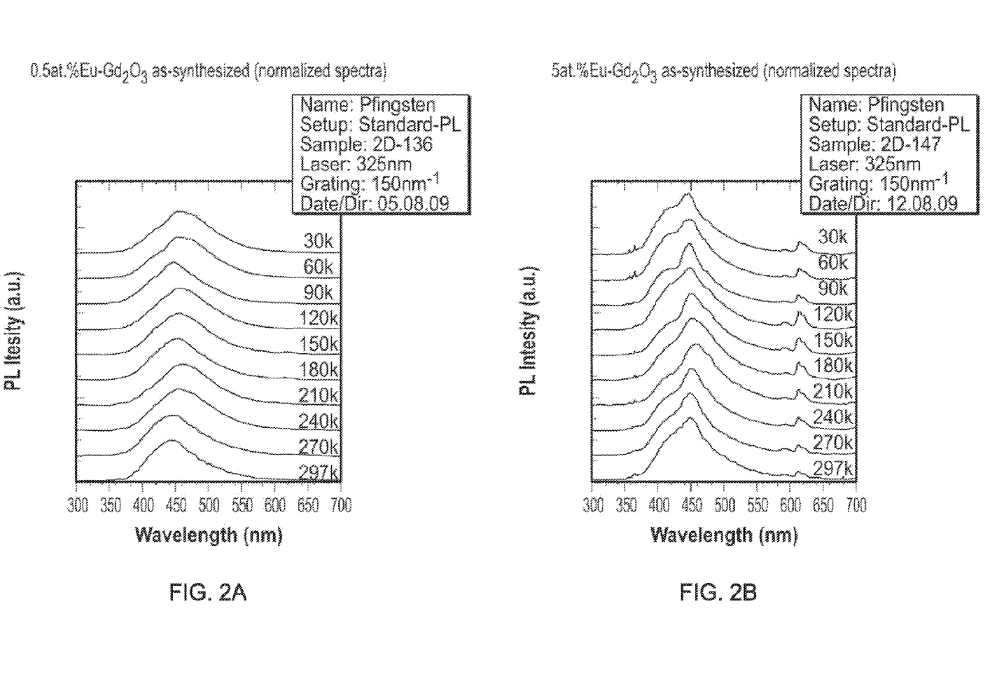

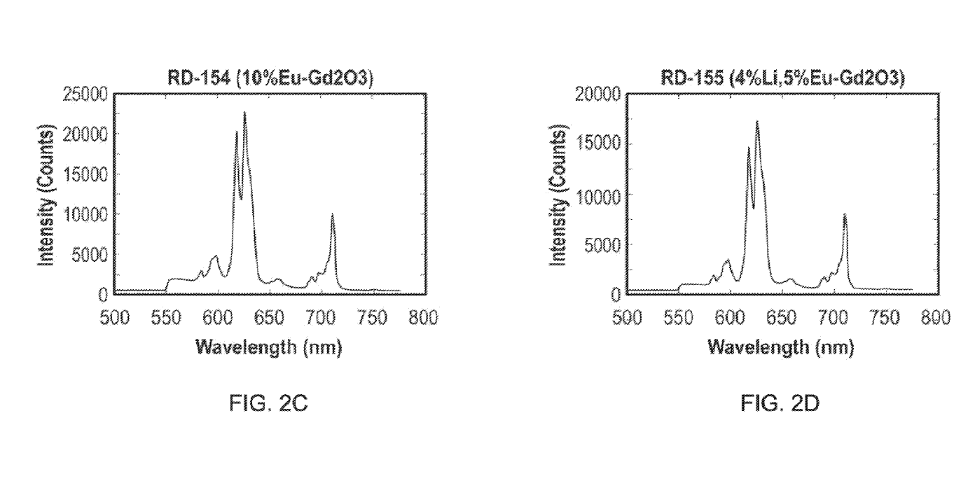

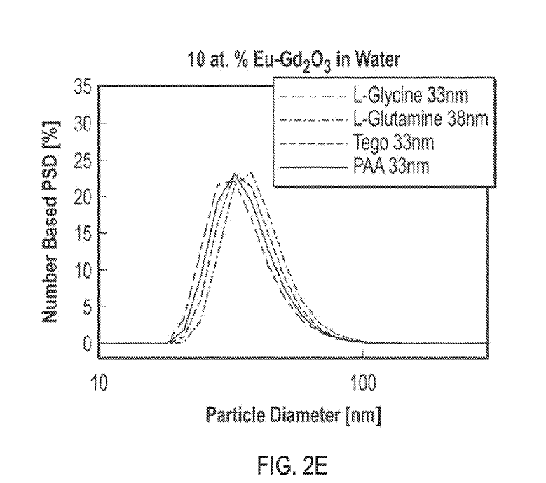

All of the characterization information is shown in Figure). This includes photoluminescence (PL) data for RD-134, RD-134S, RD-136 RD-147, RD-154, and RD-155, as well as the hydrodynamic diameter of the amino acid functionalized particles (AS-22 a-d) measured by dynamic light scattering (DLS). The PL curves for RD-134, RD-134S, RD-147, and RD-155 are taken at several temperatures from 30 K to 297 K, using an excitation wavelength of 325 nm (UV). All of the emission wavelengths are near 450 nm (blue), with the peaks appearing sharper at higher temperatures. The 1 at. % Gd—ZrO2 particles showed a small, secondary peak near 360 nm which appears to diminish somewhat as temperature increases (FIG. 2 A/B). Also, by increasing the europium doping from 0.5% to 5%, the primary peak increased in amplitude and gets sharper, and a secondary peak forms at about 610 nm (FIG. 2 C/D).

The second set of particles, RD-154 (10% Eu—Gd₂O₃) and RD-155 (4% Li/5% Eu—Gd₂O₃), had much taller, sharper PL emission peaks than the earlier specimens (FIG. 2 E/F), with a primary peak at about 625 nm (red-orange light) and a substantial secondary peak at about 710 nm (deep red light). The excitation wavelength of these particles was not shown, but is likely 325 nm. Additionally, the emission spectrum is not shown below 500 nm, but based on the very low background noise near 500 nm, there probably aren't any additional secondary peaks. Based on these results, a red-orange luminescence was expected when viewing the particles under the fluorescence microscope with UV excitation.

The amino-acid functionalized particles were characterized by measurement of hydrodynamic diameter using dynamic light scattering (DLS; FIG. 1G) in pure water. This showed diameters of 33 nm for glycine-, TEGO-, and PAA-functionalized particles, and 35 nm for glutamine-functionalized particles. These particles were believed to rapidly agglomerate in solutions of phosphate buffered saline (PBS).

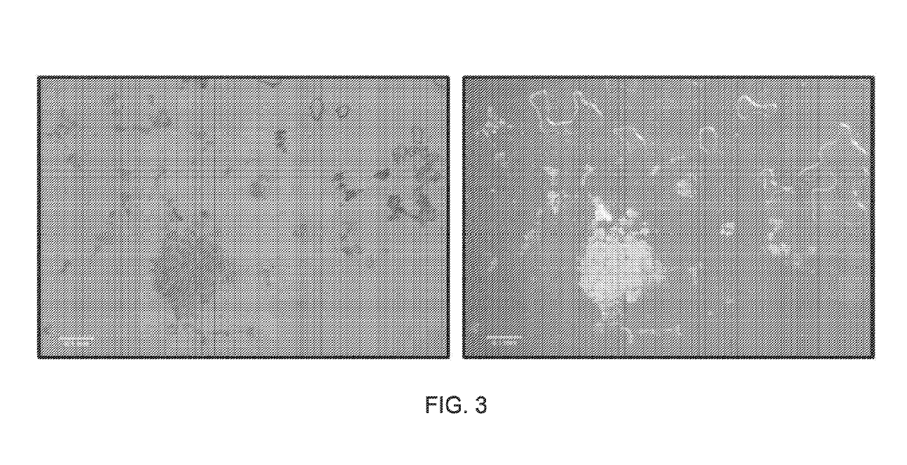

In order to observe the luminescent properties of the particles a drop of nanoparticles dissolved in PBS was placed on a microscope slide to look for any fluorescence signal. Based on the data provided, an excitation wavelength of 325 nm was expected to produce a primary peak at 450 nm, with a secondary peak caused by europium at 615 nm. The UV source present on the fluorescent microscope was more in the near-UV range, approximately 400 nm, and was not paired with any bandpass emission (barrier) filter. FIG. 3 shows a brightfield image of the particles and the same field's corresponding fluorescence image. Many of the particles did not appear to fluoresce at all, and the ones that did fluoresce emitted light across the entire visible spectrum. Based on these experiments, RD-154 (10 at. % Eu—Gd₂O₃) and RD-155 (4 at. % Li, 5 at. % Eu—Gd₂O₃) were analyzed. Both were designed to have a stronger photoluminescence than the previous particles. When viewed under UV excitation, a very large cluster of RD-154 particles did appear to show faint red fluorescence (FIG. 3). This same effect could not be seen for RD-155 (lithium-europium co-doped Gd₂O₃).

### Magnetic Resonance Imaging

After scanning several tubes of pelleted nanoparticles at T_{E}=6 ms and T_{E}=14 ms, the image sequences (in *.tif format) were viewed using ImageJ software. The sequences were subtracted from one another, and the difference image was put through the built-in background subtraction function, using a 50 pixel filter size, then cropped for viewing using MIPAV software. Once in MIPAV, quantitative analyses were performed by using the isolevel selection tool and statistics generator.

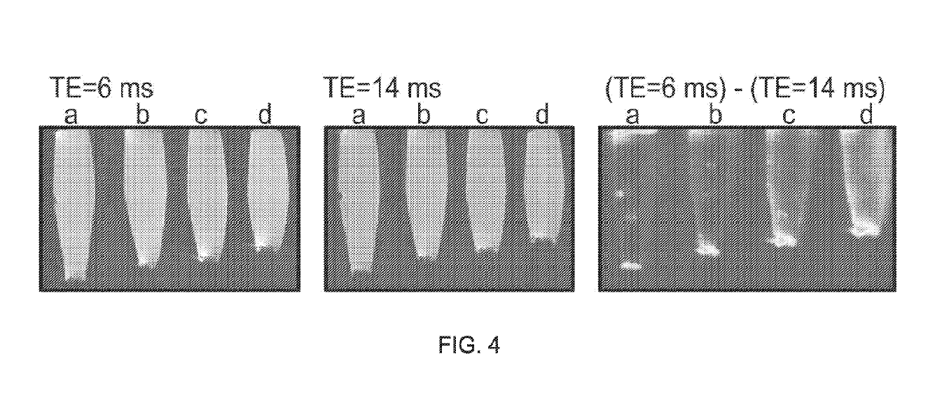

The raw scans of Eppendorf tubes containing concentrations of 0.5% Eu—Gd₂O₃ from 50 μg to 2.5 mg in 500 μL PBS, scanned at TE=6 and 14 ms, along with the resultant image achieved when subtracting the two, are shown (FIG. 4). Viewing the results in this manner, the edges of the pellet were emphasized, while the supernatant and the tube itself were reduced to near zero values. The signals from the same tubes were further enhanced by performing a 3-D rendering using the pseudocolor tool.

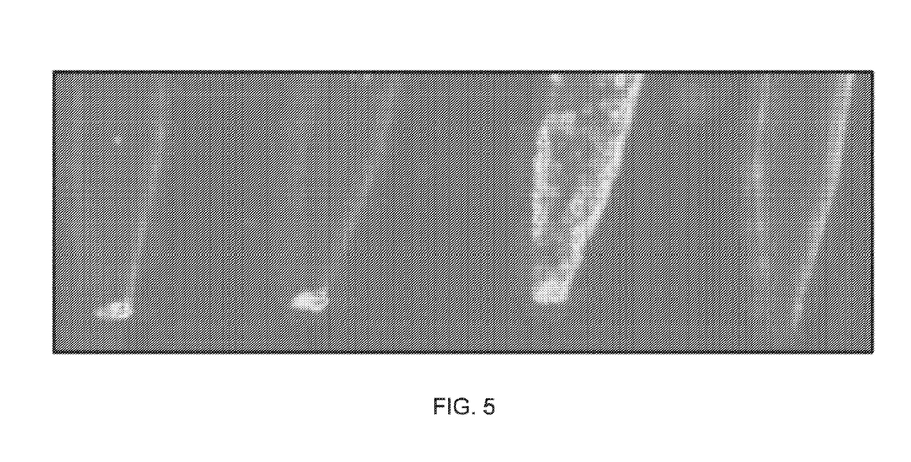

FIG. 5 shows the MRI signal for 5% Eu—Gd₂O₃. Upon processing the scans, the larger pellet (1 mg) appeared to have been disturbed sometime between centrifugation and scanning, so that a single pellet was not seen; instead, many local areas of large intensity could be seen.

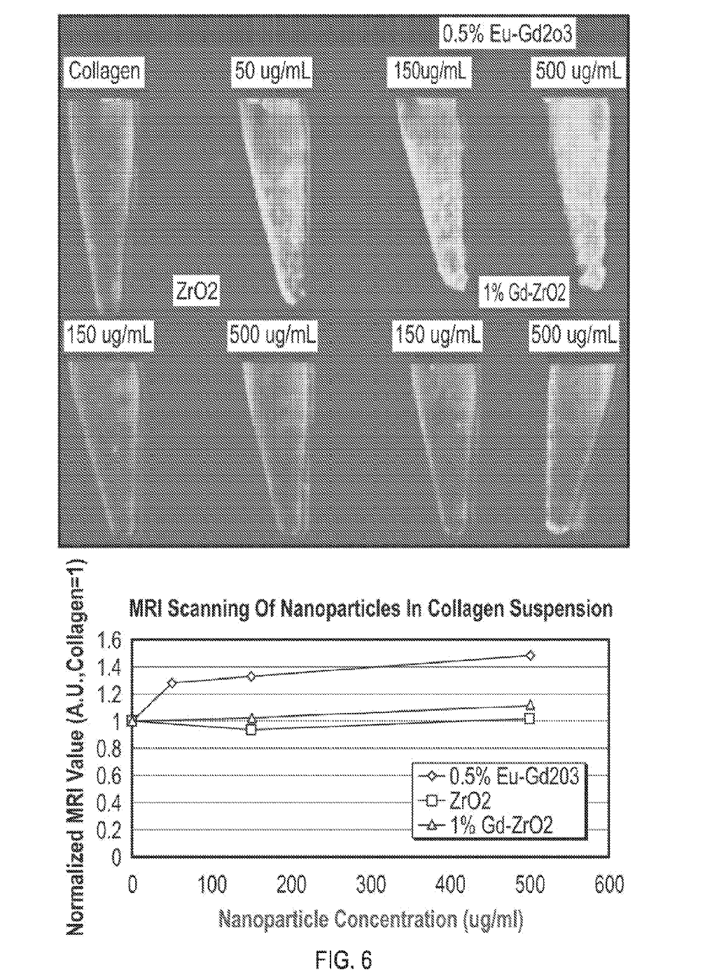

Based on the above results, it was determined that it would be beneficial to scan the particles in a more dispersed form, as they would be in native tissue. To overcome the effects of particles settling during the extended scan time, a highly viscous, concentrated solution of type I collagen derived from rat tail tendon was made into which various concentrations of nanoparticles could be suspended with less settling during the scan. Results are summarized in FIG. 6, and it can be seen that there was still some degree of settling in higher concentrations, but it can also be seen that, compared to the collagen control, tubes containing particles that give a response in MR are a different intensity throughout the entire volume. This means that the dispersion was maintained reasonably well for the duration of the scan. When the difference-subtracted images were normalized to collagen and measurements made, the resulting plots reflected the scans. The europium-doped gadolinium was about 30% higher than collagen at 50 μg/mL, and increased to about 50% higher at 500 μg/mL. Gadolinium-doped zirconium as well as zirconium oxide alone could not be differentiated from collagen by the naked eye except at very high concentrations and never deviated more than 10% from the collagen control.

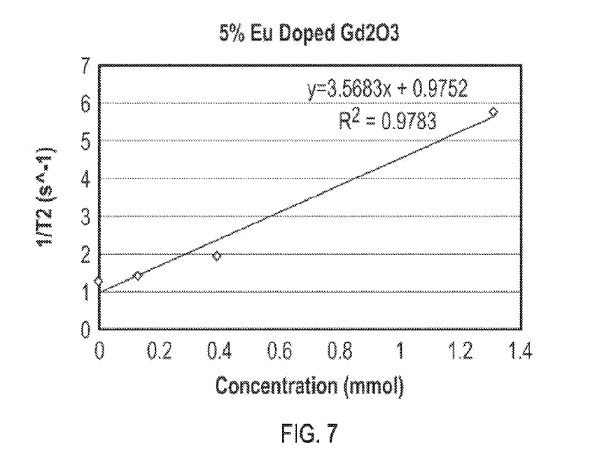

Because the 5% Eu—Gd2O3 particles had the ideal combination of MRI signal and europium doping, they were examined further. A new T2-weighted scan at several echo times (4, 6, 8, 10, 12, 14, 16, 18, and 20 ms) was performed so that the R2 relaxivity value could be calculated and compared with other available particles. The original MR images for the standardized tubes were split into individual slices which were reorganized so that each slice contained all the echo times stacked together. One by one, these were processed using the ImageJ plugin for T2-weighted images described in the methods section. The calculated T2 times were reassembled into a single stack so that MIPAV volume-of-interest (VOI) tools could be used to isolate the different concentrations within the scan. The mean T2 value was calculated for each scan, and a plot of 1/T2 vs. concentration was made (FIG. 7). The plot shows the equation of the best fit line, used to determine the R2 relaxivity value for the particles, which was 3.6 s⁻¹·mM⁻¹.

### In Vivo MRI

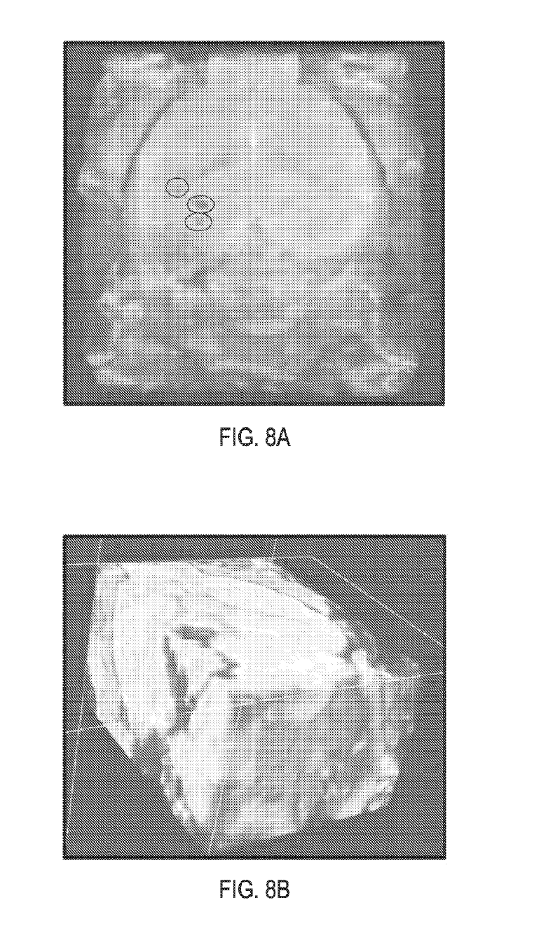

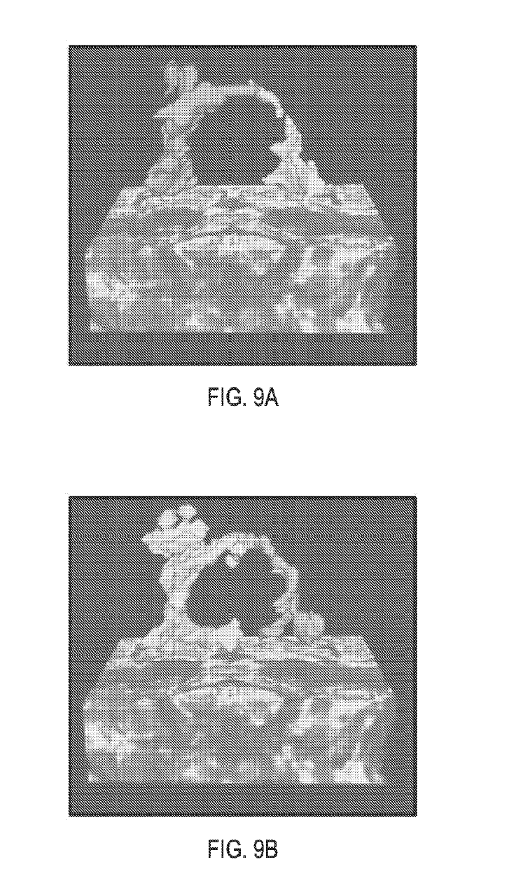

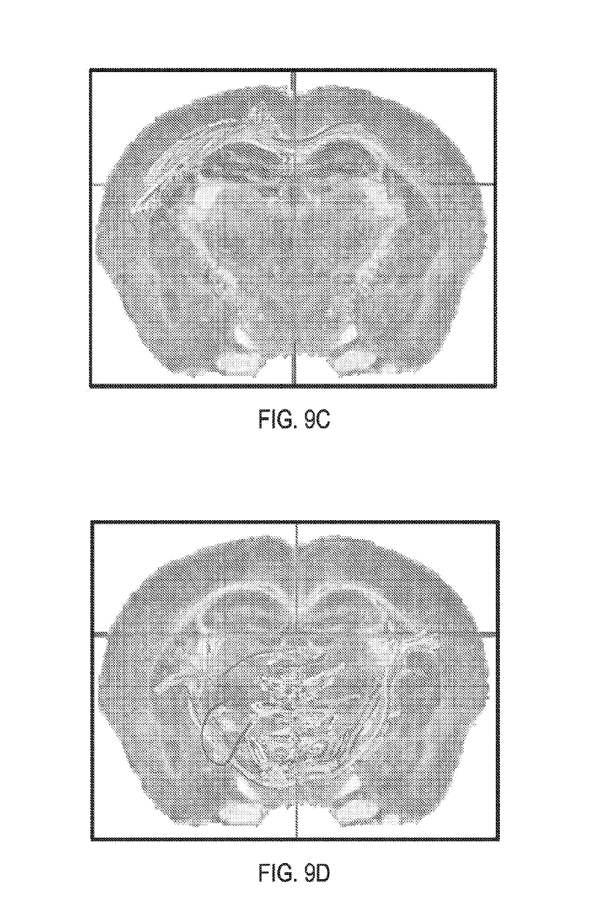

Performing in vivo microinjections of functionalized nanoparticles as described in the methods, MRI scanning one hour after the injection revealed 3 distinct injection points near the dorsal surface of the brain (FIG. 8A) which appear to have run together when looking deeper in the tissue (FIG. 8B).

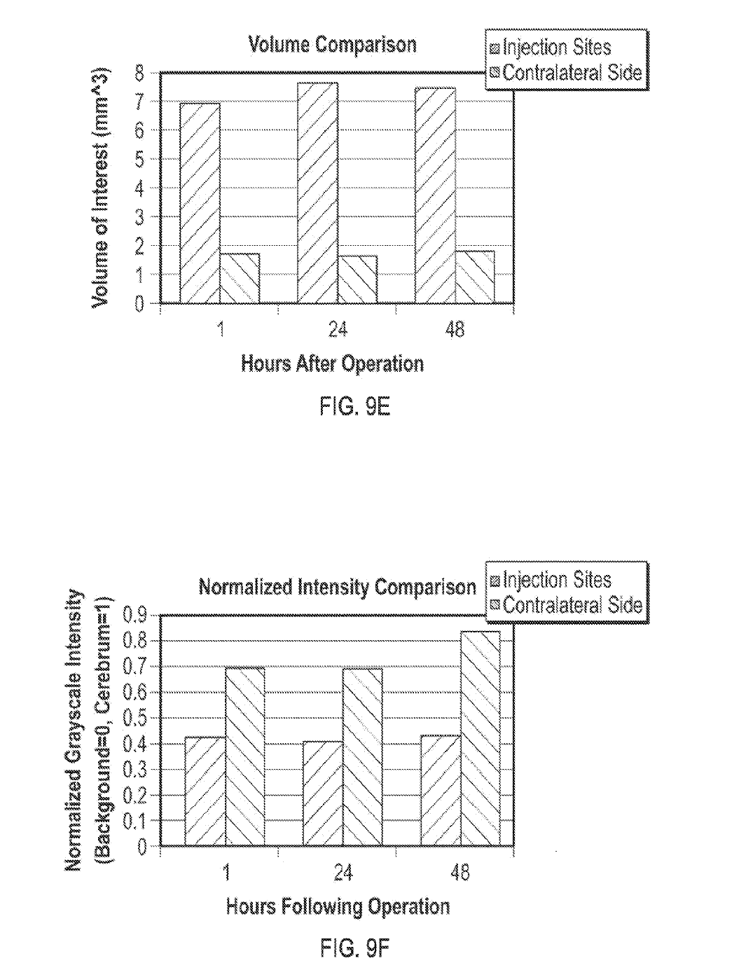

Looking at the injections collectively as one volume, all 3 sets of MRI scans were analyzed for volume and grayscale intensity. The volumes of interest (VOI) were selected by manual segmentation using MIPAV software, separately selecting both the injection sites as well as volumes of interest on the contralateral side. Qualitatively, when viewing a similarly rendered region of the brain at the 1 and 48 hour time points (FIG. 9, A-B), it appears the contrast spread slightly in the right hemisphere after 48 hours have elapsed, including an especially strong difference on the ventral side of the brain. For comparative purposes, diffusion tensor tractography for the injection area was also done on the mouse brain atlas, available at www.civm.duhs.duke.edu/neuro200902/index.html (FIG. 9, C-D). The patterns of hypointense regions appear to follow the patterns shown in the tractography for both shallow and deep injections. When attempting to quantitatively analyze the scans, the volume of the injection site VOI increased slightly, but not by a significant amount. The volume of the contralateral VOI was constant for the entire 48 hours. After normalization, so that the background level is zero, the cerebrum is 1, and every other signal is linearly interpolated, the VOI average intensity was steady for the first 24 hours before increasing slightly between 24 and 48 hours (FIG. 9, E-F). It appears the most caudal injection passed through into the cisterna ambiens, which drains into the subarachnoid space, thus allowing the particles to diffuse indirectly to the contralateral hemisphere without axonal transport.

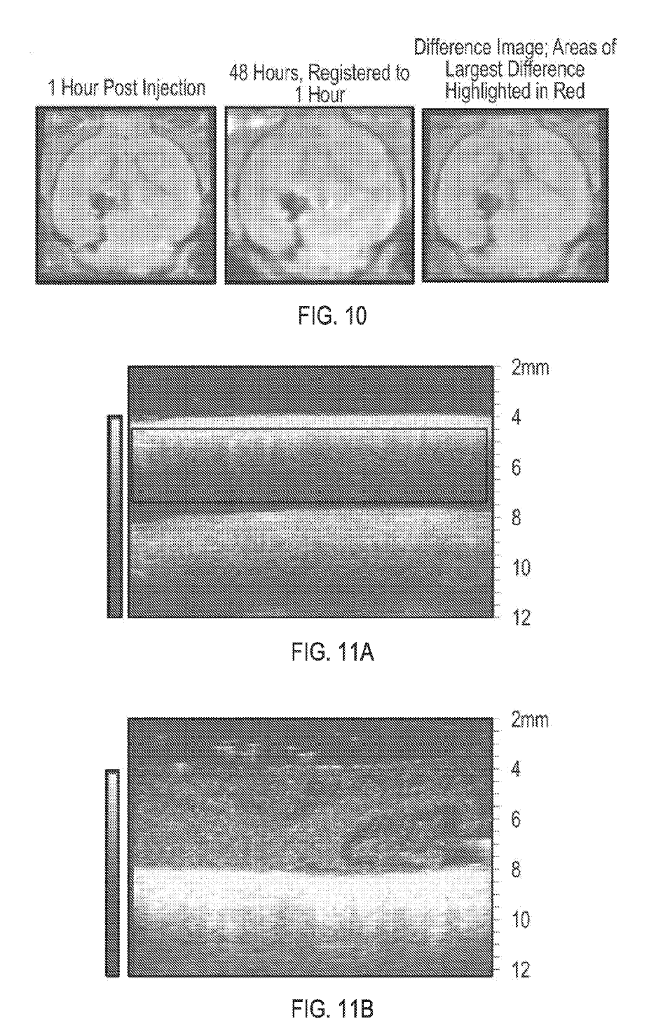

Alternatively, the data were also processed by registering the 24- and 48-hour datasets to the 1 hour dataset, using the landmark-least squares algorithm built into MIPAV. Once registration was confirmed, the differences between the datasets were found by simple pixelwise subtraction. The result of subtracting the registered 48 hour dataset from the 1 hour dataset is shown in FIG. 10. The difference image was overlaid onto the original 1 hour TE=6 ms image, showing areas of largest difference in red pixels. Regions of red are seen on the edge of the injection site, as well as in the contralateral hemisphere, showing evidence for diffusion as well as axonal transport of the particles.

### Ultrasound

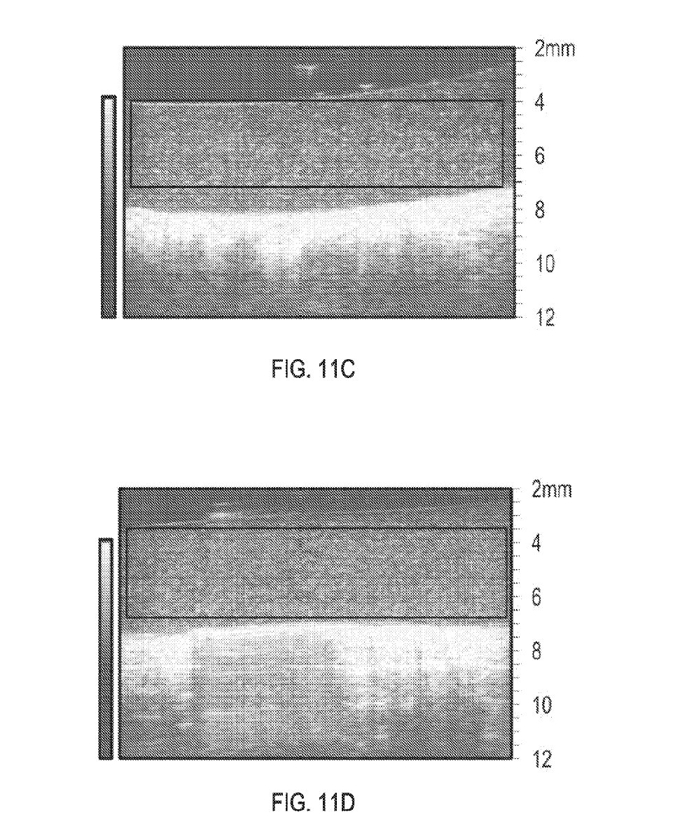

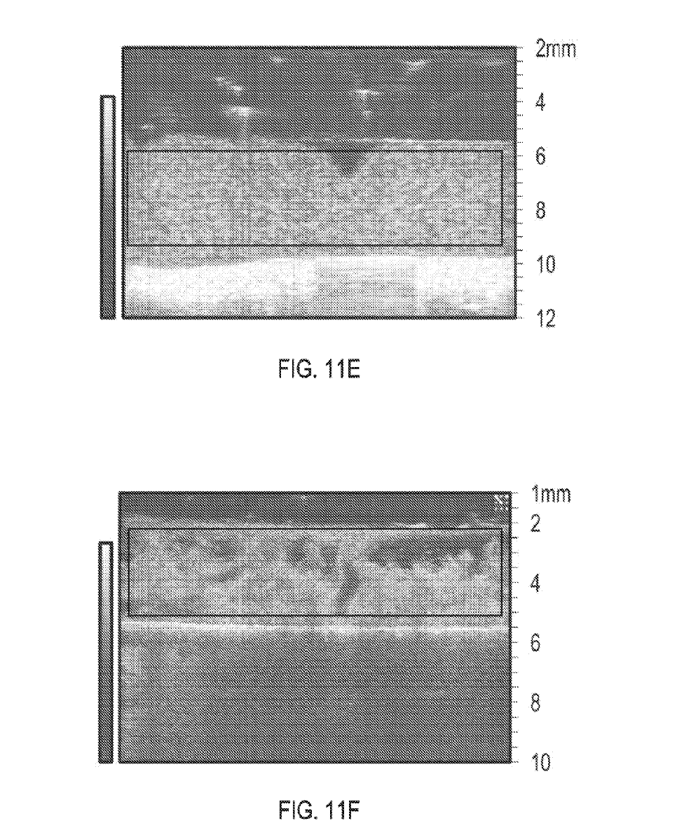

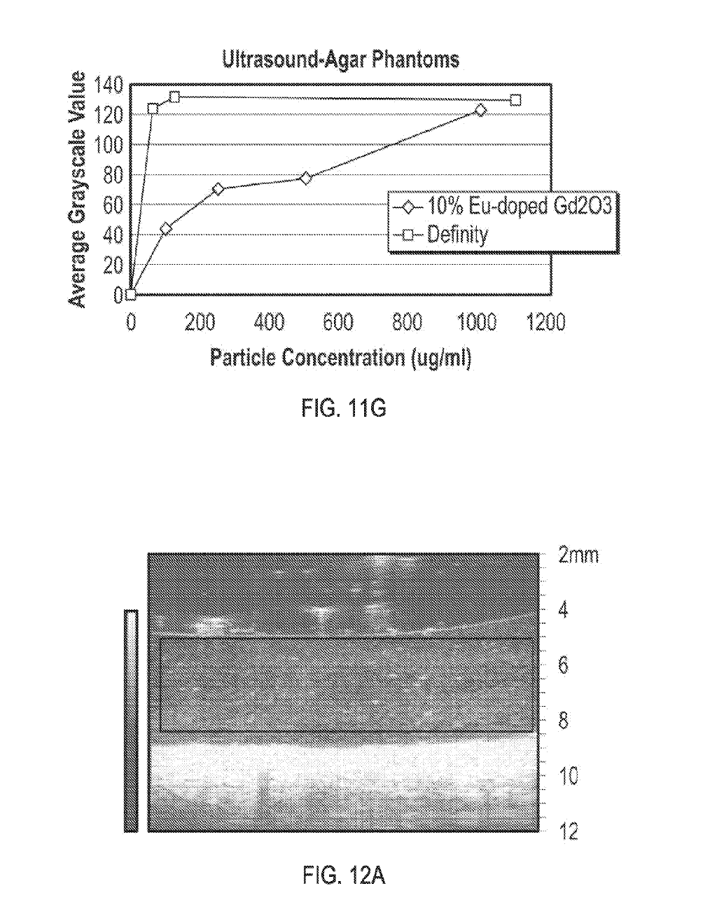

Several concentrations of 10% Eu—Gd₂O₃ suspended in agar in 60 mm Petri dishes were scanned using the 30 MHz ultrasound probe (FIG. 11). In these samples, at concentrations as low as 100 μg/mL, a difference can be seen compared with the PBS control. Further, the highest concentration is nearly as intense as Definity®, the commercially available ultrasound contrast agent used as a positive control. When shaken vigorously, the Definity® contains a hexafluoropropane concentration of about 1.1 mg/mL. The larger Definity® particles produce a more qualitative “all or none” type of detection—that is, it is difficult to distinguish between intermediate concentrations using the average grayscale measured under ultrasound (FIG. 10G). The EuGd₂O₃ particles, however, have a more linear relationship between grayscale value and concentration, meaning they may be more useful for quantitative measurements in ultrasound.

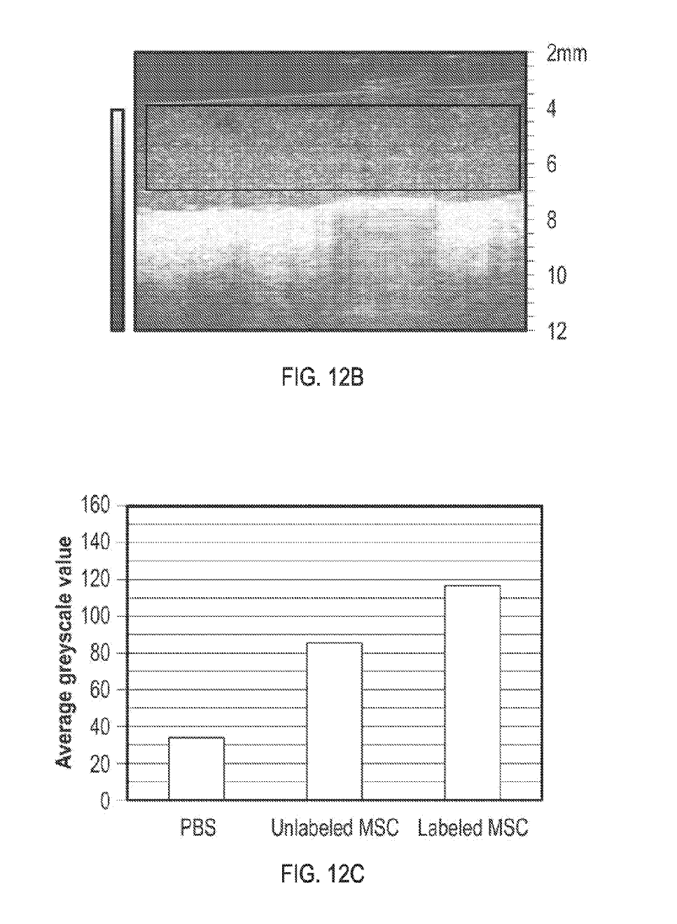

For ultrasound visualization of labeled cells, two populations of 250,000 cells each, one of which was labeled with 200 mg/mL 10% Eu—Gd₂O₃, were suspended in 60 mm Petri dishes containing warm agar and stirred until the agar set. When scanning the labeled and non-labeled cells, a difference could be seen between the two samples with the naked eye (FIG. 12).

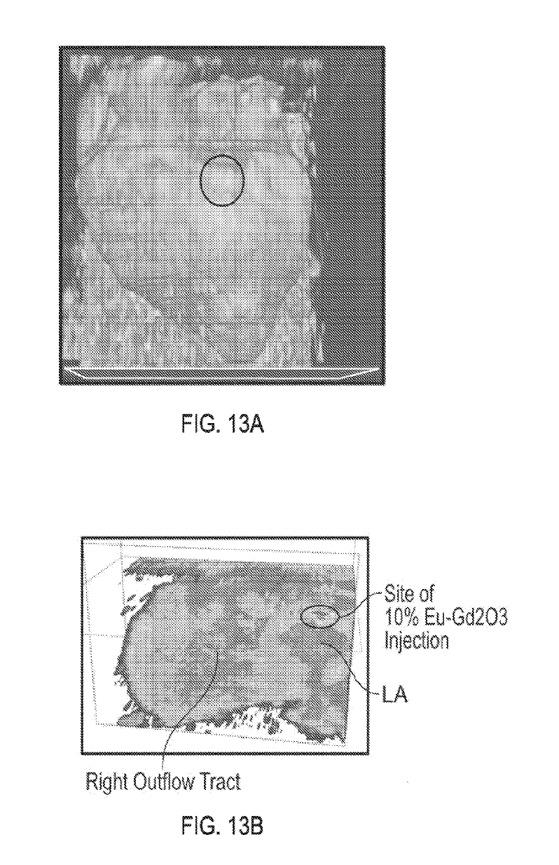

Next, an ex vivo mouse heart was scanned to gather preliminary data for in vivo experiments. After fixing the heart in 4% paraformaldehyde, 200 μg 10% Eu—Gd₂O₃ particles suspended in 20 μL volume were injected into the wall of the base of the left ventricle. To mimic imaging the heart within the body, the heart was mounted in melted agar and allowed it to set. Most of the details of the heart could be seen, and by scanning the probe across the entire heart in short axis at 30 MHz, being careful to maintain a constant probe speed while recording a cine loop, a 3-dimensional tomography could be generated (FIG. 13A). At the site of injection, a small hyperintense region could be observed with a large shadow further from the placement of the probe, indicative of a region of strong contrast (FIG. 13B).

### REFERENCES

Akhtari, M., Bragin, A., Cohen, M., Moats, R., Brenker, F., Lynch, M. D., et al. (2008). Functionalized magnetonanoparticles for MRI diagnosis and localization in epilepsy. Epilepsia, 49(8), 1419-1430.
Alric, C., Taleb, J., Le Duc, G., Mandon, C., Billotey, C., Le Meur-Herland, A., et al. (2008). Gadolinium chelate coated gold nanoparticles as contrast agents for both X-ray computed tomography and magnetic resonance imaging. Journal of the American Chemical Society, 130(18), 5908-5915.
Bara, C., Ghodsizad, A., Niehaus, M., Makoui, M., Piechaczek, C., Martin, U., et al. (2006). In vivo echocardiographic imaging of transplanted human adult stem cells in the myocardium labeled with clinically applicable CliniMACS nanoparticles. Journal of the American Society of Echocardiography: Official Publication of the American Society of Echocardiography, 19(5), 563-568.
Casciaro, S., Conversano, F., Ragusa, A., Ada Malvindi, M., Franchini, R., Greco, A., et al. (2010). Optimal enhancement configuration of silica nanoparticles for ultrasound imaging and automatic detection at conventional diagnostic frequencies. Investigative Radiology, 45:717.
Chelliah, R. K., & Senior, R. (2009). Contrast echocardiography: An update. Current Cardiology Reports, 11(3), 216-224.
Crich, S. G., Biancone, L., Cantaluppi, V., Duo, D., Esposito, G., Russo, S., et al. (2004). Improved route for the visualization of stem cells labeled with a gd-/Eu-chelate as dual (MRI and fluorescence) agent. Magnetic Resonance in Medicine: Official Journal of the Society of Magnetic Resonance in Medicine/Society of Magnetic Resonance in Medicine, 51(5), 938-944.
Eder, M., Becker, K., Rammes, G., Schierloh, A., Azad, S. C., Zieglgansberger, W., et al. (2003). Distribution and properties of functional postsynaptic kainate receptors on neocortical layer V pyramidal neurons. The Journal of Neuroscience: The Official Journal of the Society for Neuroscience, 23(16), 6660-6670.
Fedele, E., & Foster, A. C. (1992). 3H]glycine uptake in rat hippocampus: Kinetic analysis and autoradiographic localization. Brain Research, 572(1-2), 154-163.
Frank, J. A., Miller, B. R., Arbab, A. S., Zywicke, H. A., Jordan, E. K., Lewis, B. K., et al. (2003). Clinically applicable labeling of mammalian and stem cells by combining superparamagnetic iron oxides and transfection agents. Radiology, 228(2), 480-487.
Groman, E. V., Bouchard, J. C., Reinhardt, C. P., & Vaccaro, D. E. (2007). Ultrasmall mixed ferrite colloids as multidimensional magnetic resonance imaging, cell labeling, and cell sorting agents. Bioconjugate Chemistry, 18(6), 1763-1771.
Mallidi, S., Joshi, P. P., Sokolov, K., & Emelianov, S. (2009). On sensitivity of molecular specific photoacoustic imaging using plasmonic gold nanoparticles. Conference Proceedings: . . . Annual International Conference of the IEEE Engineering in Medicine and Biology Society. IEEE Engineering in Medicine and Biology Society. Conference, 2009, 6338-6340.
Pietsch, H., Jost, G., Frenzel, T., Raschke, M., Walter, J., Schirmer, H., et al. (2009). Efficacy and safety of lanthanoids as X-ray contrast agents. European Journal of Radiology,
Regino, C. A., Walbridge, S., Bernardo, M., Wong, K. J., Johnson, D., Lonser, R., et al. (2008). A dual CT-MR dendrimer contrast agent as a surrogate marker for convection-enhanced delivery of intracerebral macromolecular therapeutic agents. Contrast Media & Molecular Imaging, 3(1), 2-8.
Rice, H. E., Hsu, E. W., Sheng, H., Evenson, D. A., Freemerman, A. J., Safford, K. M., et al. (2007). Superparamagnetic iron oxide labeling and transplantation of adipose-derived stem cells in middle cerebral artery occlusion-injured mice. AJR. American Journal of Roentgenology, 188(4), 1101-1108.
Sandmann, Alice, Ruzica Djenadic, Ahmed Khalil, and Markus Winterer. “Colloidal Dispersions: Surface Chemistry and Particle Size.” Nanoparticle Process Technology. University of Duisburg Essen, Feb. 4, 2012. Web. 5 Jun. 2012.
Slowing, I. I., Trewyn, B. G., & Lin, V. S. (2007). Mesoporous silica nanoparticles for intracellular delivery of membrane-impermeable proteins. Journal of the American Chemical Society, 129(28), 8845-8849.
Tallheden, T., Nannmark, U., Lorentzon, M., Rakotonirainy, O., Soussi, B., Waagstein, F., et al. (2006). In vivo MR imaging of magnetically labeled human embryonic stem cells. Life Sciences, 79(10), 999-1006.
Zhang, X. L., Sullivan, J. A., Moskal, J. R., & Stanton, P. K. (2008). A NMDA receptor glycine site partial agonist, GLYX-13, simultaneously enhances LTP and reduces LTD at schaffer collateral-CA1 synapses in hippocampus. Neuropharmacology, 55(7), 1238-1250.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

1. A method for imaging in a mammal, comprising:
a) providing a mammal administered a composition comprising Eu—Gd₂O₃ nanoparticles;
b) applying ultrasound or laser pulses to the mammal and recording images of the Eu—Gd₂O₃ nanoparticles in the mammal; and
c) analyzing the images or applying x-rays or a magnetic field to the mammal and recording images of the Eu—Gd₂O₃ nanoparticles in the mammal.

2. (canceled)

3. The method of claim 1 further comprising
administering to the mammal the composition comprising Eu—Gd₂O₃ nanoparticles; and
applying the x rays or a magnetic field.

4. A method to image a device introduced to a mammal, comprising:
a) introducing to a mammal a device having embedded therein Eu—Gd₂O₃ nanoparticles;
b) applying ultrasound, laser pulses, x-rays or a magnetic field to the mammal; and
c) detecting the location of the device in the mammal.

5. The method of claim 1 wherein the Eu—Gd₂O₃ nanoparticles are about 1 nm to about 50 nm in diameter.

6. The method of claim 1 wherein the Eu—Gd₂O₃ nanoparticles are less than about 200 nm in diameter.

7. The method of claim 1 wherein the Eu—Gd₂O₃ nanoparticles further comprise a targeting agent.

8. The method of claim 7 wherein the agent is an antibody or an antigen binding portion thereof.

9. The method of claim 1 wherein the images are of a heart, lung, kidney, liver, pancreas, bladder, ovary, uterus, or brain.

10. The method of claim 1 wherein the composition is injected into the mammal.

11. The method of claim 10 wherein the composition is subcutaneously, intradermally or intravascularly injected into the mammal.

12. The method of claim 1 wherein the composition is locally administered.

13. The method of claim 1 wherein the mammal is a human.

14. The method of claim 1 wherein an ultrasound image or a photoacoustic image is recorded before a magnetic resonance image.

15. (canceled)

16. The method of claim 1 wherein an ultrasound image or a photoacoustic image is recorded before an x-ray image.

17. (canceled)

18. The method of claim 4 wherein the device is a lead, catheter or needle.

19. The method of claim 1 wherein the nanoparticles further comprise a moiety that enhances biocompatibility.

20. The method of claim 1 wherein the nanoparticles further comprise a cell surface ligand.

21-24. (canceled)

25. A kit comprising a device and Eu—Gd₂O₃ nanoparticles.

26. The kit of claim 25 wherein the device comprises the nanoparticles.

27. The kit of claim 25 wherein the nanoparticles are packaged separately from the device.

28. The kit of claim 26 wherein the nanoparticles are in a physiologically compatible fluid.

29. (canceled)

30. The kit of claim 25 wherein the device is a syringe, catheter, stent or lead.
